(19) United States
(12) Patent Application Publication
(10) Pub. No.: US 2012/0015049 A1
(43) Pub. Date: Jan. 19, 2012

(54) MICRORNA BIOMARKER IN CANCER

(76) Inventor: Lin Zhang, Philadelphia, PA (US)

(21) Appl. No.: 13/144,685

(22) PCT Filed: Jan. 14, 2010
(86) PCT No.: PCT/US2010/021047

§ 371 Date: Sep. 28, 2011

Related U.S. Application Data
(60) Provisional application No. 61/144,650, filed on Jan. 14, 2009.

(51) Int. Cl. A61K 31/7105; C12Q 1/68; C07H 21/00; A61P 35/00; C40B 40/06; A61K 31/713; A61K 33/24; C40B 30/00; C40B 30/04

(52) U.S. Cl. 424/649; 506/7; 435/6.11; 435/6.13; 536/24.5; 506/9; 506/16; 536/24.3; 514/44 A; 514/44 R

(57) ABSTRACT

This invention provides compositions and methods for predicting and improving a chemotherapy response to treat an ovarian cancer. In one embodiment, the invention provides compositions and methods for detecting the expression level of Let-7i microRNA to predict a chemotherapy response. In another embodiment, the invention provides compositions and methods for enhancing the expression level of Let-7i microRNA to improve a chemotherapy response.

### CROSS REFERENCE TO RELATED APPLICATIONS

This application claims priority to U.S. Provisional Patent Application 61/144,650, filed Jan. 14, 2009, which is incorporated by reference herein in its entirety.

### STATEMENT OF GOVERNMENT RIGHTS

The work described herein was supported, in part, by a grant from the National Cancer Institute of the NIH, grant number P50-CA083638. The United States government may have certain rights in the invention.

### FIELD OF THE INVENTION

The invention relates to compositions and methods for predicting and improving a chemotherapy response to treat an ovarian cancer. Specifically, the invention relates to detecting the expression level of Let-7i microRNA to predict a chemotherapy response and enhancing the expression level of Let-7i microRNA to improve the chemotherapy response.

### BACKGROUND OF THE INVENTION

Epithelial ovarian cancer is the most frequent cause of gynecologic malignancy-related mortality in women. Although advances in platinum-based chemotherapy have resulted in improved survival, patients typically experience disease relapse within two years of initial treatment and develop platinum resistance. Therefore, development of new therapies is a high priority. Molecular targeted drugs hold promise as independent therapeutic agents or as chemotherapy response modifiers and could contribute substantial improvements to the outlook of women with ovarian cancer. So far, the studies in the identification of druggable targets and biomarkers for ovarian cancer have thus far mainly focused on the role of protein-coding genes, whereas our knowledge of functional noncoding genomic sequences, such as microRNAs (miRNAs), is still in its infancy.

MicroRNAs (miRNAs) are ˜22 nucleotide non-coding RNAs, which negatively regulate gene expression in a sequence-specific manner. Up to one-third of human messenger RNAs (mRNAs) appear to be miRNA targets. Each miRNA can target hundreds of transcripts directly or indirectly, while more than one miRNA can cover a single transcript target. Therefore, the potential regulatory circuitry afforded by miRNA is enormous. Increasing evidence indicates that miRNAs are key regulators of various fundamental biological processes. Let-7 is among the founding and best understood miRNAs. In organisms such as mouse, rat, and human, the let-7 family is composed of multiple members with overlapping or distinct functions. Eleven members of let-7 have been identified in the human genome. Most importantly, the let-7 family is one of the first reported tumor suppressor miRNAs in cancer, which negatively regulates the RAS and is expressed at lower levels in lung tumors than in normal lung tissue. Although the let-7 family has been generally shown to be a tumor suppressor gene, there have been contradictory reports that it can serve an oncogenic function.

A need exists to understand the mechanisms of various miRNAs in ovarian cancer treatments in order to develop improved methods for diagnosis, prognosis, and treatment of ovarian cancer.

### SUMMARY OF THE INVENTION

In one embodiment, the invention provides a method for determining a chemotherapy response to treat a cancer, in a subject, comprising the steps of: obtaining a biological sample from said subject; and testing said biological sample to determine whether or not an miRNA is under-expressed in said biological sample, relative to the expression of said miRNA in a control sample, whereby the under-expression of said miRNA in said biological sample indicates a tumor response to said chemotherapy. In one exemplary embodiment, said miRNA is Let-7i. In another exemplary embodiment, said cancer is an ovarian cancer.

In another embodiment, the invention provides a method for diagnosis of a cancer, in a subject, the method comprising the steps of: obtaining a biological sample from said subject; and testing said biological sample to determine whether or not an miRNA is under-expressed in said sample, relative to the expression of said miRNA in a control sample, whereby the under-expression of said miRNA in said biological sample indicates that a tumor in said subject is resistant to a chemotherapy. In one exemplary embodiment, said miRNA is Let-7i. In another exemplary embodiment, said cancer is an ovarian cancer.

In another embodiment, the invention provides a method of providing a prognosis for a cancer, in a subject, the method comprising the steps of: obtaining a biological sample from said subject; and testing said biological sample to determine whether or not an miRNA is under-expressed in said sample, relative to the expression of said miRNA in a control sample, whereby the under-expression of said miRNA in said biological sample indicates that a tumor in said subject is resistant to a chemotherapy. In one exemplary embodiment, said miRNA is Let-7i. In another exemplary embodiment, said cancer is an ovarian cancer.

In another embodiment, the invention provides a method of treating a cancer, in a subject, the method comprising the steps of: determining whether or not an miRNA is under-expressed in said sample, relative to the expression of said miRNA in a control sample, whereby the under-expression of said miRNA in said biological sample indicates that a tumor in said subject is resistant to a chemotherapy; and thereby selecting a treatment method for said cancer. In one exemplary embodiment, said miRNA is Let-7i. In another exemplary embodiment, said cancer is an ovarian cancer.

In another embodiment, the invention provides a method of improving a chemotherapy response to a cancer treatment, in a subject, the method comprising administering an effective amount of an agent that enhances the expression of an miRNA. In an exemplary embodiment, said agent is a oligonucleotide based pre-mir-Let-7 drug.

In another embodiment, the invention provides a method of treating a cancer, in a subject, the method comprising: administering an effective amount of a chemotherapy agent and an effective amount of an agent that enhances the expression of an miRNA. In an exemplary embodiment, the agent that enhances the expression of an miRNA is a oligonucleotide based pre-mir-Let-7 drug.

In another embodiment, the invention provides a method for determining a survival of a subject with an ovarian cancer, the method comprising the steps of: obtaining a biological sample from said subject; and determining the expression level of Let-7i, whereby the expression level of Let-7i in said biological sample indicates survivability of said subject.

In another embodiment, the invention provides a kit for determining a chemotherapy response in a patient with a cancer, said kit comprising: a) a oligonucleotide complementary to an miRNA; and b) optionally, reagents for the formation of the hybridization between said oligonucleotide and said miRNA. In one exemplary embodiment, said miRNA is Let-7i. In another exemplary embodiment, said cancer is an ovarian cancer.

In another embodiment, the invention provides an apparatus for determining a chemotherapy response in a patient with a cancer, said apparatus comprising a solid support, wherein a surface of said solid support is linked to an oligonucleotide complementary to an miRNA. In one exemplary embodiment, said miRNA is Let-7i. In another exemplary embodiment, said cancer is an ovarian cancer. In yet another exemplary embodiment, said apparatus is a micro-array.

In another embodiment, the invention provides a pharmaceutical composition for improving a tumor response to chemotherapy, said composition comprising an effective amount of an agent that enhances the expression of an miRNA in said tumor. In one exemplary embodiment, said miRNA is Let-7i. In another exemplary embodiment, said agent is a oligonucleotide based pre-mir-Let-7 drug.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE FIGURES

FIG. 1 shows that Let-7i expression is significantly reduced in patients with chemotherapy-resistant EOC. A, microarray analysis of miRNA expression between complete response (CR) and noncomplete response (non-CR) ovarian cancer patients. B, differentially expressed miRNAs between complete response and noncomplete response patients at various statistical significance (P<0.015, P<0.025, and P<0.05). C, validation of Let-7i expression in complete response and noncomplete response patients by real-time reverse transcription-PCR.

FIG. 2 shows that Let-7i expression regulates cis-platinum resistance of EOC cells. A, inhibition of Let-7i, but not mir-509-3p or mir-509-5p, increased resistance to cis platinum treatment in 2008 and SKOV3 cells. B, stem-loop real-time reverse transcription-PCR showed endogenous Let-7i was significantly blocked by Let-7i inhibitor. C, overexpression of Let-7i by retroviral infection in 2008, SKOV3, and MCF7 cells increased their sensitivity to the cis-platinum treatment. Inset, stem-loop real-time reverse transcription-PCR showed that Let-7i was stably overexpressed in EOC cell lines by retroviral transfection.

FIG. 3 shows that Let-7i DNA copy number does not exhibit genomic alteration in human cancer. Genomic locus harboring Let-7i did not exhibit alteration in EOC (n=106). Black, deletion; gray, amplification.

FIG. 4 shows that low Let-7i expression is significantly associated with shorter survival of patients with EOC. Correlation between Let-7i expression and survival of EOC patients were analyzed by microarray (A, progression-free survival), real-time reverse transcription-PCR (B, progression-free survival), and tissue array (C, disease-free survival).

FIG. 5 illustrates a potential mechanism of Let-7i regulating chemotherapy sensitivity in human cancer.

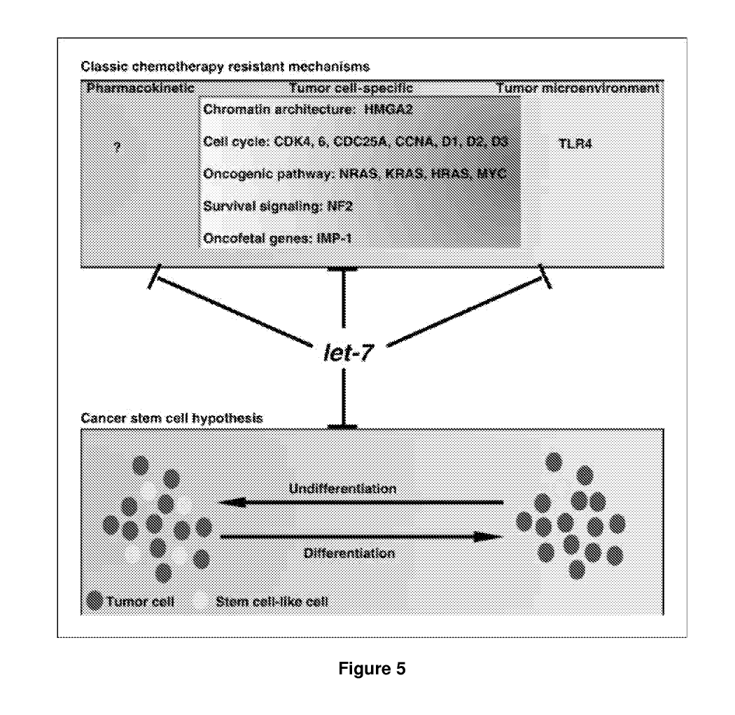

FIG. 6 shows that Let-7 mimic treatment inhibits tumor cell growth in vitro. Tumor cell lines (A2780, 2008, SKOV3 (ovarian); SKBR3, MCF7 (breast); and HeLa (cervical).) were treated with let-7 mimic or control oligos in vitro. Cell growth was measured by MTT assay (Roche). The proliferating rates of the let-7 mimic treated cells (red/black) were significantly lower than the control cells (green/grey) after 72 hrs.

FIG. 7 shows that Let-7 mimic treatment increases chemotherapy sensitivity in vitro. Tumor cell lines (A2780, 2008, SKOV3 (ovarian); SKBR3, MCF7 (breast); and HeLa (cervical).) were treated with chemotherapy drug cisplatinum and let-7 mimic or control oligos in vitro. Cell growth was measured by MTT assay (Roche). The combination therapy (platinum+let-7 mimic) significantly reduced tumor cell growth.

### DETAILED DESCRIPTION OF THE INVENTION

The invention relates to compositions and methods for predicting and improving a chemotherapy response to treat an ovarian cancer. Specifically, the invention relates to detecting the expression level of an miRNA to predict a chemotherapy response and enhancing the expression level of the miRNA to improve the chemotherapy response.

In one embodiment, the expression level of an miRNA associated with a chemotherapy response is measured in a biological sample. In an exemplary embodiment, the miRNA associated with a chemotherapy response is a Let-7 miRNA. Examples of Let-7 miRNA include, but are not limited to, Let-7a, Let-7b, Let-7c, Let-7d, and Let-7i. In a particular embodiment, the Let-7 miRNA is Let-7i.

The biological sample can be a tissue, blood, or other biological sample known to one of skill in the art. In one example, a tissue sample can be removed from a subject in accordance with a method known to one of skill in the art. In another example, a blood sample can be removed from a subject, and white blood cells can be isolated for extraction of nucleic acids by standard techniques.

In one embodiment, a control sample is obtained from a subject whose tumor positively responds to a chemotherapy treatment. Examples of a positive tumor response include, but are not limited to, reduction in tumor size, reduction in tumor growth rate, cessation of further tumor growth, non-proliferation of tumor cells, and death of tumor cells. The expression level of an miRNA in the control sample is determined, and in one embodiment, such expression level serves as a control expression level of the miRNA. The expression level of an miRNA in a test sample obtained from a treatment subject, relative to its expression level in the control sample, is indicative of a response to chemotherapy.

In one embodiment, the expression level of an miRNA in a test sample is greater than the expression level of the miRNA in a control sample (i.e., expression of the miRNA gene product is “over-expressed”). As used herein, the expression of an miRNA is “over-expressed” when the amount of miRNA expression in a test sample from a subject is greater than the amount of the expression level of the miRNA in a control sample. In another embodiment, the expression level of an miRNA in a test sample is less than the expression level of the miRNA in a control sample (i.e., expression of the miR gene product is “under-expressed”). As used herein, the expression of an miRNA is “under-expressed” when the amount of miRNA expression in a test sample from a subject is less than the amount of the expression level of the miRNA in a control sample. In yet another embodiment, the expression level of an miRNA in a test sample is equal to the expression level of the miRNA expression in a control sample. The relative miRNA expression in the control and normal samples can be determined with respect to one or more RNA expression standards.

The level of an miRNA in a sample can be measured using any technique that is suitable for detecting RNA expression levels in a biological sample. Suitable techniques for determining RNA expression levels in cells from a biological sample are well known to those of skill in the art. Examples of such techniques include, but are not limited to, Northern blot analysis, RT-PCR, microarrays, in situ hybridization. In a particular embodiment, a high-throughput system, for example, a microarray, is used to measure the expression level of a plurality of genes.

In one embodiment, the level of an miRNA is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question.

Suitable probes for Northern blot hybridization of a given miRNA can be produced from the nucleic acid sequences of the miRNA. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11.

In one example, the nucleic acid probe can be labeled with, e.g., a radionuclide, such as 3H, 32P, 33P, 14C, or 35S; a heavy metal; or a ligand capable of functioning as a specific binding pair member for a labeled ligand (e.g., biotin, avidin or an antibody), a fluorescent molecule, a chemiluminescent molecule, or an enzyme. Probes can be labeled to high specific activity by nick translation, random priming, or other methods known to one of skill in the art. For example, by replacing preexisting nucleotides with highly radioactive nucleotides according to the nick translation method, it is known to prepare 32P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of miRNA transcript levels. In another embodiment, miRNA gene transcript levels can be quantified by computerized imaging systems, such the Molecular Dynamics 400-B 2D Phosphorimager available from Amersham Biosciences, Piscataway, N.J.

In another embodiment, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In another embodiment, determining the levels of an miRNA expression can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the disclosure of which is incorporated herein by reference.

The relative number of miRNA gene transcripts in cells can also be determined by reverse transcription of miRNA gene transcripts, followed by amplification of the reverse-transcribed transcripts by polymerase chain reaction (RT-PCR). The levels of miRNA gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art. In another embodiment, a high throughput stem loop real-time quantitative polymerase chain reaction (RT-qPCR) is used to detect miRNA expression. See Mestdagh et al., Nucleic Acid Research, 2008, vol. 36, No. 21e143.

In some instances, it may be desirable to simultaneously determine the expression level of a plurality of different miRNA gene products in a sample. In other instances, it may be desirable to determine the expression level of the transcripts of all known miRNAs correlated with a cancer. In one embodiment, assessing cancer-specific expression levels for hundreds of miRNAs requires a large amount of total RNA (e.g., 20 μg for each Northern blot) and autoradiographic techniques that require radioactive isotopes.

In another embodiment, an oligolibrary, in microchip format (i.e., a microarray), may be constructed containing a set of probe oligodeoxynucleotides that are specific for a set of miRNA genes. Using such a microarray, the expression level of multiple miRNAs in a biological sample can be determined by reverse transcribing the RNAs to generate a set of target oligodeoxynucleotides, and hybridizing them to probe oligodeoxynucleotides on the microarray to generate a hybridization, or expression, profile. The hybridization profile of the test sample can then be compared to the pre-determined expression level of a control sample to determine which miRNAs have an altered expression level in cancer cells. As used herein, “probe oligonucleotide” or “probe oligodeoxynucleotide” refers to an oligonucleotide that is capable of hybridizing to a target oligonucleotide. “Target oligonucleotide” or “target oligodeoxynucleotide” refers to a molecule to be detected (e.g., via hybridization). By “miRNA-specific probe oligonucleotide” or “probe oligonucleotide specific for an miRNA” is meant a probe oligonucleotide that has a sequence selected to hybridize to a specific miRNA gene product, or to a reverse transcript of the specific miRNA gene product.

An “expression profile” or “hybridization profile” of a particular sample is essentially a fingerprint of the state of the sample; while two states may have any particular gene similarly expressed, the evaluation of a number of genes simultaneously allows the generation of a gene expression profile that is unique to the state of the cell. That is, normal tissue may be distinguished from a cancer tissue, and within a cancer tissue, different prognosis states (good or poor long term survival prospects, for example) may be determined. By comparing expression profiles of a cancer tissue in different states, information regarding which genes are important (including both up- and down-regulation of genes) in each of these states is obtained. The identification of sequences that are differentially expressed in a cancer tissue or normal tissue, as well as differential expression resulting in different prognostic outcomes, allows the use of this information in a number of ways. For example, a particular treatment regime may be evaluated (e.g., to determine whether a chemotherapeutic drug act to improve the long-term prognosis in a particular patient). Similarly, diagnosis may be done or confirmed by comparing patient samples with the known expression profiles. Furthermore, these gene expression profiles (or individual genes) allow screening of drug candidates that suppress the cancer expression profile or convert a poor prognosis profile to a better prognosis profile.

The microarray can be prepared from gene-specific oligonucleotide probes generated from known miRNA sequences. In one embodiment, the array contains two different oligonucleotide probes for each miRNA, one containing the active, mature sequence and the other being specific for the precursor of the miRNA. The array may also contain controls, such as one or more mouse sequences differing from human orthologs by only a few bases, which can serve as controls for hybridization stringency conditions. tRNAs from both species may also be printed on the microchip, providing an internal, relatively stable, positive control for specific hybridization. One or more appropriate controls for non-specific hybridization may also be included on the microchip. For this purpose, sequences are selected based upon the absence of any homology with any known miRNAs.

The microarray may be fabricated using techniques known in the art. For example, probe oligonucleotides of an appropriate length are 5′-amine modified at position C6 and printed using commercially available microarray systems, e.g., the GENEMACHINE, OMNIGRID 100 MICROARRAYER and AMERSHAM CODELINK activated slides. Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6×SSPE/30% formamide at 25° C. for 18 hours, followed by washing in 0.75×TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary miRNA, in the patient sample. According to one embodiment, the labeled cDNA oligomer is a biotin-labeled cDNA, prepared from a biotin-labeled primer. The microarray is then processed by direct detection of the biotin-containing transcripts using, e.g., STREPTAVIDIN-ALEXA647 conjugate, and scanned utilizing conventional scanning methods. Image intensities of each spot on the array are proportional to the abundance of the corresponding miRNA in the patient sample.

In addition to use for quantitative expression level assays of a specific miRNA, a microchip containing miRNA-specific probe oligonucleotides corresponding to a substantial portion of the miRNome, preferably the entire miRNome, may be employed to carry out miRNA gene expression profiling, for analysis of miRNA expression patterns. Distinct miRNA signatures can be associated with established disease markers, or directly with a disease state.

According to the expression profiling methods described herein, total RNA from a sample from a subject suspected of having a cancer (e.g., ovarian cancer) is quantitatively reverse transcribed to provide a set of labeled target oligodeoxynucleotides complementary to the RNA in the sample. The target oligodeoxynucleotides are then hybridized to a microarray comprising miRNA-specific probe oligonucleotides to provide a hybridization profile for the sample. The result is a hybridization profile for the sample representing the expression pattern of miRNA in the sample. The hybridization profile comprises the signal from the binding of the target oligodeoxynucleotides from the sample to the miRNA-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a control sample. An alteration in the signal is indicative of a chemotherapy response in the subject.

Other techniques for measuring miRNA gene expression are also within the skill in the art, and include various techniques for measuring rates of RNA transcription and degradation.

In another embodiment, the invention provides a method for prognosis of a cancer. The method comprises the step of determining whether or not an miRNA is over-expressed or under-expressed in a sample, relative to the expression of the same miRNA in a control sample. In some embodiments, the over-expression or under-expression of the miRNA indicates a tumor response to a chemotherapy, and thereby provides a prognosis for a cancer. In one embodiment, the under-expression of an miRNA indicates that the tumor is resistant to a chemotherapy. In one exemplary embodiment, said miRNA is Let-7i. In another exemplary embodiment, said cancer is an ovarian cancer.

In another embodiment, the invention provides a kit for predicting response to a chemotherapy in a patient with a cancer, said kit comprising: a) a oligonucleotide complementary to an miRNA; and b) optionally, reagents for the formation of the hybridization between said oligonucleotide and said miRNA. In another embodiment, the kit optionally includes directions for monitoring the nucleic acid molecule levels of a marker in a biological sample derived from a subject. In another embodiment, the kit comprises a sterile container which contains the primer, probe, or other detection regents; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the primers or probes described herein and their use in diagnosing a cancer. Preferably, the kit further comprises any one or more of the reagents described in the diagnostic assays described herein. In other embodiments, the instructions include at least one of the following: description of the primer or probe; methods for using the enclosed materials for the diagnosis of a cancer; precautions; warnings; indications; clinical or research studies; and/or references. The instructions may be printed directly on the container (when present), or as a label applied to the container, or as a separate sheet, pamphlet, card, or folder supplied in or with the container.

In another embodiment, the invention provides an apparatus for determining a chemotherapy response in a patient with a cancer, said apparatus comprising a solid support, wherein a surface of said solid support is linked to an oligonucleotide complementary to an miRNA. In one embodiment, the apparatus is a micro-array. The examples of solid support include, but are not limited to, a glass or nitro-cellulose slide that is used to bind nucleic acids.

In another embodiment, the invention provides a method of treating a cancer, in a subject, the method comprising the steps of: obtaining a biological sample from said subject; and determining whether or not an miRNA is under-expressed in said sample, relative to the expression of said miRNA in a control sample, whereby the under-expression of said miRNA in said biological sample indicates that a tumor in said subject is resistant to a chemotherapy; thereby selecting a treatment method for said cancer. In one embodiment, said miRNA is a Let-7 miRNA. In a particular embodiment, said miRNA is Let-7i.

In another embodiment, the invention provides a method of treating a cancer, the method comprising administering an effective amount of an agent that enhances the expression of an microRNA. In one embodiment, said miRNA is a Let-7 miRNA. In a particular embodiment, said miRNA is Let-7i. In another embodiment, the agent is a shRNA from a polymerase II or III promoter. In another embodiment, the agent is a double-stranded miRNA mimic. In another embodiment, the agent is an oligonucleotide based pre-mir-Let-7 drug.

The terms “treat”, “treating” and “treatment”, as used herein, refer to ameliorating symptoms associated with a disease or condition, for example, an ovarian cancer, including preventing or delaying the onset of the disease symptoms, and/or lessening the severity or frequency of symptoms of the disease or condition. The terms “subject” and “individual” are defined herein to include animals, such as mammals, including but not limited to, primates, cows, sheep, goats, horses, dogs, cats, rabbits, guinea pigs, rats, mice or other bovine, ovine, equine, canine, feline, rodent, or murine species. In a preferred embodiment, the mammal is a human.

As used herein, an “effective amount” of an isolated miRNA is an amount sufficient to inhibit proliferation of a cancer cell in a subject suffering from a cancer. One skilled in the art can readily determine an effective amount of an miRNA gene product to be administered to a given subject, by taking into account factors, such as the size and weight of the subject; the extent of disease penetration; the age, health and sex of the subject; the route of administration; and whether the administration is regional or systemic.

Cancers that may be treated by the invention include tumors that are not vascularized, or not yet substantially vascularized, as well as vascularized tumors. The cancers may be comprised of non-solid tumors (such as leukemias and lymphomas) or may be solid tumors.

Types of cancers treated with the agent or composition of the invention include carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers alike may be treated in accordance with the invention.

Examples of tumors/cancers which may be treated include, ovarian, breast (including HER2+ and metastatic), colorectal, colon, renal, rectal, pancreatic, prostate, stomach, gastrointestinal, gastric, stomach, esophageal, bile duct, lung (including small cell and non-small cell lung tumors; adenocarcinoma of the lung and squamous carcinoma of the lung), liver, epidermoid tumors, squamous tumors such as head and neck tumors, epithelial squamous cell cancer, thyroid, cervical, neuroendocrine tumors of the digestive system, neuroendocrine tumors, cancer of the peritoneum, hepatocellular cancer, hepatoblastoma, HPCR, glioblastoma, bladder cancer, hepatoma, endometrial or uterine carcinoma, salivary gland carcinoma, kidney or renal cancer, bone cancer, soft tissue sarcoma (including embryonal and alveolar rhabdomyosarcoma, GIST, alveolar soft part sarcoma and clear cell sarcoma), cholangiocarcinoma, bile cancer, gallbladder carcinoma, myeloma, vulval cancer, hepatic carcinoma, anal carcinoma, penile carcinoma, retinal, hematopoietic cancer, androgen-dependent tumors, androgen-independent tumors, Other examples include Kaposi's sarcoma, synovial sarcoma, vasoactive intestinal peptide secreting tumor, CNS neoplasms, neuroblastomas, capillary hemangioblastomas, meningiomas, and cerebral metastases, melanoma, rhabdomyosarcoma, glioblastoma, including glioblastoma multiforme, EMB, RMS, ALV, medulloblastoma, ependymoma, Wilm's cancer, Ewing's cancer, osteosarcoma, PNT, rhabdoid, rhabdomyosarcoma, retinoblastoma, adrenal cortical cancer, adrenal cancer, and leiomyosarcoma. In a particular embodiment, the cancer treated by the invention is human epithelial ovarian cancer.

In another embodiment, the invention provides a method of improving a chemotherapy response to a cancer treatment, in a subject, the method comprising the steps of: detecting an expression level of an miRNA to determine whether or not said miRNA is under-expressed in said sample, relative to the miRNA expression in a control sample, whereby the under-expression of said miRNA in said biological sample indicates that a tumor in said subject is resistant to a chemotherapy; and administering an effective amount of an agent that enhances the expression of said miRNA. In an exemplary embodiment, said miRNA is Let-7i.

In another embodiment, the invention provides a method of improving a chemotherapy response to a cancer treatment, in a subject, the method comprising administering an effective amount of an agent that enhances the expression of an microRNA. In one embodiment, said miRNA is Let-7i. In another embodiment, the agent is a shRNA from a polymerase II or III promoter. In another embodiment, the agent is a double-stranded miRNA mimic. miRNA mimic technology is well known in the art. See e.g., Wang, Z., 2009, miRNA mimic technology, In MicroRNA Interference Technologies, pages 93-100, Springer-Link Publications. In another embodiment, the agent is an oligonucleotide based pre-mir-Let-7 drug.

Polynucleotide therapy featuring a polynucleotide encoding an miRNA is another therapeutic approach for enhancing a transcript number or expression level of the miRNA in a subject. Expression vectors encoding the miRNAs can be delivered to cells of a subject for the treatment or prevention of a cancer. The nucleic acid molecules are delivered to the cells of a subject in a form in which they can be taken up and are advantageously expressed so that therapeutically effective levels can be achieved.

Methods for delivery of the polynucleotides to the cell according to the invention include using a delivery system, such as liposomes, polymers, microspheres, gene therapy vectors, and naked DNA vectors.

Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding an miRNA molecule can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:31 1-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No.5,399,346). Non-viral approaches can also be employed for the introduction of a miRNA therapeutic to a cell of a patient diagnosed as having a neoplasia. For example, an miRNA can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512. 1983), asialoorosoinucoid-polylysine conjugation. (Wu el at. Journal of Biological Chemistry 263:14621, 1988; Wu et al., Journal of Biological Chemistry 264:16985, 1989), or by micro-injection under surgical conditions (Wolff et al., Science 247:1465 1990), Preferably the rnicroRNA molecules are administered in combination with a liposome and protamine,

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Micro RNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. For any particular subject, the specific dosage regimes should be adjusted over time according to the individual need and the professional judgment of the person administering or supervising the administration of the compositions.

In another embodiment, the invention provides therapeutic compositions that increase the expression of a microRNAs described herein for the treatment or prevention of a cancer or for improving a chemotherapy response. In another embodiment, the present invention provides a pharmaceutical composition comprising an agent that enhances the expression of an miRNA of the invention. Polynucleotides of the invention may be administered as part of a pharmaceutical composition. The composition is preferably sterile and contains a therapeutically effective amount of a polynucleotide molecule in a unit of weight or volume suitable for administration to a subject.

The therapeutic polynucleotide molecule described herein may be administered with a pharmaceutically-acceptable carrier, in unit dosage form. Conventional pharmaceutical practice may be employed to provide suitable formulations or compositions to administer the compounds to patients suffering from a cancer.

Carrier as used herein includes pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN.®., polyethylene glycol (PEG), and PLURONICS.®.

The active ingredients may also be entrapped in microcapsules prepared, for example, by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes. Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT.®. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

In another embodiment, the compositions of the invention are administered in conjunction with other therapeutic agents. In another embodiment, the compositions of the invention are administered in conjunction with radiotherapy, chemotherapy, photodynamic therapy, surgery or other immunotherapy, to a patient who has a hyperproliferative disorder, such as cancer or a tumor. In one example, the compositions of the present invention are administered to a patient in conjunction with chemotherapy, radiation therapy, or both chemotherapy and radiation therapy. The compositions of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional antibodies, or other therapeutic agents.

Examples of chemotherapeutic agents include, but are not limited to, platinum analogs such as cisplatin and carboplatin; vinblastine; platinum; proteins such as arginine deiminase and asparaginase; alkylating agents such as thiotepa and cyclosphosphamide (CYTOXAN.™); alkyl sulfonates such as busulfan, improsulfan and piposulfan; androgens such as calusterone, dromostanolone propionate, epitiostanol, mepitiostane, testolactone; anti-adrenals such as aminoglutethimide, mitotane, trilostane; anti-androgens such as flutamide, nilutamide, bicalutamide, leuprolide, and goserelin; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston); anti-metabolites such as methotrexate and 5-fluorouracil (5-FU); folic acid analogues such as denopterin, methotrexate, pteropterin, trimetrexate; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; folic acid replenisher such as frolinic acid; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; taxanes, e.g. paclitaxel (TAXOL.®., Bristol-Myers Squibb Oncology, Princeton, N.J.) and docetaxel (TAXOTERE.®., Rhne-Poulenc Rorer, Antony, France); topoisomerase inhibitor RFS 2000; thymidylate synthase inhibitor (such as Tomudex); additional chemotherapeutics including aceglatone; aldophosphamide glycoside; aminolevulinic acid; amsacrine; be strabucil; bisantrene; edatrax ate; defofamine; demecolcine; diaziquone; difluoromethylornithine (DMFO); elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PS K.®.; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacyto sine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; xeloda; ibandronate; CPT-11; retinoic acid; esperamicins; and capecitabine.

Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for inhibitory nucleic acid molecules include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

### EXAMPLES

### Materials and Methods

Patients and specimens. All frozen ovarian cancer specimens used in this study were collected at the University of Turin, Turin, Italy. Clinical characteristics were as previously defined and listed in Table 1. Optimal surgical debulking was ≦1 cm of residual individual tumor nodules. Front-line chemotherapy comprised platinum, platinum-cyclophosphamide, or (after 1995) platinum-paclitaxel. Complete response to therapy was defined by normalization of physical examination, abdomino-pelvic computerized tomography (CT) scan and serum CA-125. Noncomplete response included partial response (≧50% decrease in the sum of greater tumor dimensions by CT) and no response (<50% decrease or any increase in tumor). Progression-free survival was the time between completion of chemotherapy and first recurrence (if a complete response had been achieved) or progression of disease, defined as ≧50% tumor increase by CT scan or two increasing CA-125 values. All tumors were from primary sites, and were immediately snap-frozen and stored at −80° C. Tissues were obtained after patients' written consent under a general tissue collection protocol approved by the Institutional Review Board of the University of Pennsylvania and the University of Turin.

Cell lines and cell culture. Ovarian (SKOV3, 2008, OVCAR10, OVCAR3), cervical (HeLa), and breast (MCF7, MDA-MB-468) cancer cell lines were cultured in RPMI 1640 (Invitrogen) supplemented with 10% fetal bovine serum and 1% antibiotics (Invitrogen).

RNA isolation. Total RNA was isolated from 100 to 500 mg of frozen tissue or 1×10⁶ cultured cells with TRIzol reagent (Invitrogen). The quality and quantity of the isolated RNA was analyzed using a Bioanalyzer 2100 system (Agilent).

miRNA microarray. miRNA microarray was performed as previously described. Briefly, 5 μg of total RNA was reverse-transcribed using biotin end-labeled random-octamer oligonucleotide primer. Hybridization of biotin-labeled complementary DNA was performed on the Ohio State University miRNA microarray chip (OSU_CCC version 3.0), which contains 1,100 miRNA probes, including 326 human miRNA genes, spotted in duplicates. Often, more than one probe exists for a given mature miRNA. Additionally, there are quadruplicate probes corresponding to most pre-miRNAs. The hybridized chips were washed and processed to detect biotin containing transcripts by STREPTAVIDIN-ALEXA 647 conjugate and scanned on an AXON 4000B microarray scanner (Axon Instruments).

Microarray analysis. The normalized microarray data were managed and analyzed by GENESPRING (Agilent), GENEPATTERN, 10 BRB-ARRAYTOOLS version 3.6,11 and microarray software suite 4 (TM4). JAVA TREEVIEW 1.0 (Stanford University School of Medicine, Stanford, Calif.) was used for tree visualization.

Stem-loop real-time reverse transcription-PCR (TaqMan miRNA assay). Expression of mature miRNAs was analyzed by TAQMAN miRNA Assay (Applied Biosystems) under conditions defined by the supplier. Briefly, single-stranded cDNA was synthesized from 5.5 ng of total RNA in a 15 μL reaction volume using the TAQMAN MICRORNA Reverse Transcription kit (Applied Biosystems). The reactions were first incubated at 16° C. for 30 min, then at 42° C. for 30 min. The reactions were inactivated by incubation at 85° C. for 5 min. Each cDNA generated was amplified by quantitative PCR using sequence-specific primers from the TAQMAN MICRORNA ASSAYS HUMAN PANEL on an Applied Biosystems 7900HT sequence detection system (Applied Biosystems). The 20 μL PCR included 10 μL of 2×Universal PCR Master Mix (no AmpErase UNG), 2 μL of each 10×TAQMAN MICRORNA ASSAY MIX and 1.5 μL of reverse transcription product. The reactions were incubated in a 384-well plate at 95° C. for 10 min, followed by 40 cycles at 95° C. for 15 s and 60° C. for 1 min.

Retroviral transduction and stable cell line generation. The retrovirus-based human miRNA expression vector was purchased from GENESERVICE. Retroviral vector containing human let-7i or control vector was transfected into the packing cell line PT67 (Clontech) using FUGENE6 Transfection Reagent (Roche). The medium was changed 48 h post transfection and the medium containing retrovirus was collected 48 h later. Human tumor cells were infected with retrovirus in the presence of 8 μg/mL of polybrene.

Transfection of inhibitor oligos. miRIDIAN inhibitors and negative controls were purchased from Dharmacon. Cells were seeded in a 96-well or 24-well plate in antibiotic-free medium to reach a 40% to 50% confluence the next day. Twenty-four hours later, the medium was replaced prior to transfection. Transfection was performed using LIPOFECTAMINE 2000 transfection reagent (Invitrogen) following the instructions of the manufacturer. For 24-well plates, the concentration used for inhibitors was 80 nmol/L, and for 96-well plates, the concentration used for inhibitors was 66 nmol/L. Cells were incubated in the medium containing the transfection mixture for 72 h until RNA extraction (from 24-well plate) or 3-(4,5-dimethylthiazol-2-yl)-2,5-diphenyltetrazolium bromide (MTT) assay (in 96-well plates) was performed.

Cis-platinum treatment. Cells were seeded in a 96-well plate in antibiotic-free medium. cis-Diamineplatinum(II) dichloride (Sigma) or mock Dulbecco' s PBS alone was added into the medium at various concentrations. The MTT assay was performed 72 h post drug addition.

MTT assay. MTT assay was performed in a 96-well plate using the CELL PROLIFERATION KIT (I) (Roche) following the manufacturer's instructions. Four to six wells were done for each sample and experiments were repeated twice. The resulting colored solution was quantified using an EMAX precision microplate reader (Molecular Devices) at 570 nm with a reference wavelength of 650 nm.

Tissue microarray. The tissue microarray was constructed as described previously. In brief, tumors were embedded in paraffin and 5-μm sections were stained with H&E to select representative regions for biopsies. Four core tissue biopsies were obtained from each specimen. The presence of tumor tissue on the arrayed samples was verified on H&E-stained sections. The patient material consisted of 53 primary ovarian carcinomas with serous histology only. The patients were treated at the Helsinki University Central Hospital between 2000 and 2004. Patients who became disease-free after the primary treatment (surgery and platinum-taxane-based chemotherapy) were included in the study, and disease-free survival was the time from diagnosis to relapse of the disease.

miRNA in situ hybridization and image analysis. In situ detection of miRNA expression was performed on formalin-fixed paraffin-embedded tissue microarray sections. Slides were deparaffinized in xylene series and rehydrated through an ethanol series (100% to 25%). After proteinase K digestion (30 μg/mL; Roche) for 10 min and postfixation in 4% paraformaldehyde, slides were prehybridized in hybridization solution (50% formamide, 5×SSC, 500 Ag/mL yeast tRNA, 1×Denhardt' s solution) for 1 h and hybridized overnight with digoxigenin-labeled miRNA-locked nucleic acid probe (EXIQON) in hybridization solution. After stringent washes (50% formamide, 2×SSC) at hybridization temperature, chromogenic detection of signals was performed using anti-digoxigenin antibody (Roche, 1:400 dilution) and PowerVision+Poly-HRP IHC detection kit (ImmunoVision Technologies) according to the manufacturer's instructions. Occasionally, a nuclear signal was seen most likely representing nonspecific staining as it was also seen in the negative controls. Therefore, only cytoplasmic staining (mature miRNA) of the tumor cells was recorded and classified as positive or negative without knowledge of the patient outcome.

Array-based comparative genomic hybridization. BAC clones included in the “1 Mb array” platform were used. Briefly, 4,134 clones from the CalTech A/B and RPCI-11 libraries were collected from both commercial and private sources and were mapped to build 34 of the human genomes using either an STS-marker (29%), end sequences (68%), or full sequences (3%). A minimum of two replicates per clone were printed on each slide. One microgram of tumor and reference DNA was labeled with Cy3 or Cy5, respectively (Amersham) using the BIOPRIME random-primed labeling kit (Invitrogen). In parallel experiments, tumor DNA and reference DNA were labeled with the opposite dye to account for differences in dye incorporation and to provide additional data for analysis. A systematic protocol was used to analyze array-based comparative genomic hybridization (aCGH) data for copy number alterations. For quality control purposes, clones demonstrating an adjusted foreground-to-background intensity ratio of <0.8 in the reference channel were removed. With dye swap data merged as input, copy number breakpoints were estimated for each sample by the Circular Binary segmentation algorithm using breakpoint significance based on 10,000 permutations. Additional analyses and visualization of aCGH data were done using the CGHAnalyzer software suite.

Statistical analysis. Statistical analysis was performed using the SPSS statistics software package (SPSS). All results were expressed as mean±SD, and P<0.05 was used for significance. Kaplan-Meier curves were used to estimate 5-year survival rates and were compared with the use of log rank statistics.

Example 1

### Let-7i Expression Is Significantly Reduced in Patients with Chemotherapy-Resistant EOC

To identify miRNA expression signatures associated with resistance to chemotherapy in patients with EOC, specimens from 72 late-stage (stage III and IV) patients were initially analyzed by miRNA microarray. A total of 69 patients with well-documented chemotherapy response information were included for further biomarker identification, and all (n=72) were used for survival analysis. The clinical characteristics of those patients are listed in Table 1.

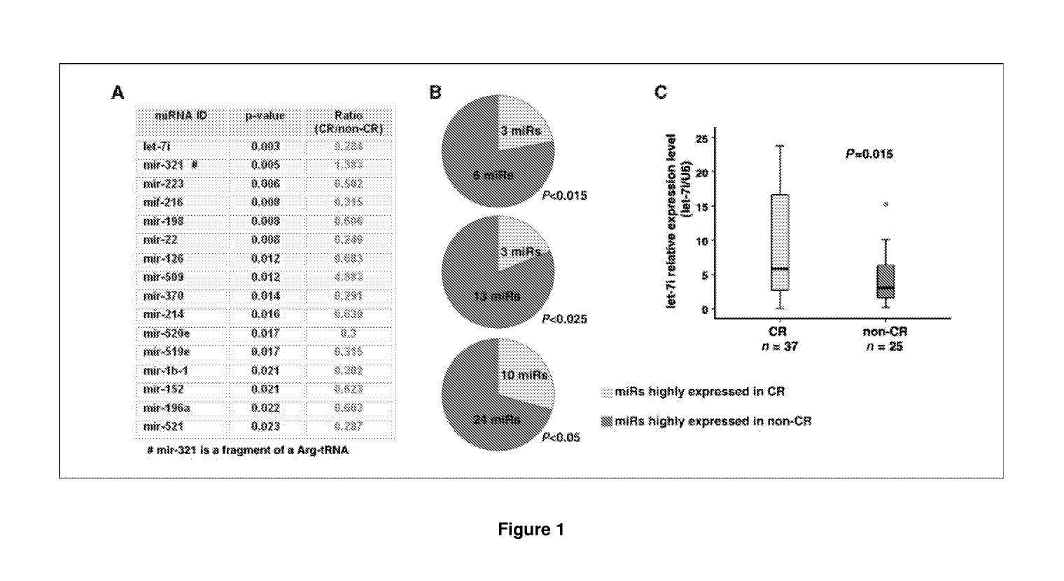

First, differences in miRNA expression between the complete response (n=42) and noncomplete response groups (n=27, including partial response and no response) were analyzed. It was found that 34 miRNAs were statistically different (P<0.05) between the groups, with 24 (70.6%) miRNAs higher in the noncomplete response group and 10 miRNAs (29.4%) higher in the complete response group. Importantly, nine miRNAs exhibited even greater statistical significance (P<0.015) and of those, six were higher in the noncomplete response group and three were higher in the complete response group (FIGS. 1A and B). In particular, Let-7i, a tumor suppressor miRNA, was the top differential miRNA between the two groups and expressed at remarkably lower levels in the noncomplete esponse group (expression ratio of complete response roup to noncomplete response group=9.3, P=0.003, n=69; FIG. 1A). To further validate this finding, Let-7i expression was examined in 62 randomly selected late-stage EOC specimens by stem-loop real-time reverse transcription-PCR. Consistent with the microarray data, Let-7i expression was indeed significantly reduced in the noncomplete response patients (9.1±1.5 relative expression unit; Let-7i/U6; n=25) as compared with their counterparts with complete response (4.3±0.7 relative expression unit; Let-7i/U6; n=37, P=0.015; FIG. 1C). In addition, this result was further confirmed in EpCAM-positive tumor cells isolated from the ascites of late-stage ovarian cancer patients (˜13.9-fold higher in enriched tumor cells from the complete chemotherapy response patients compared with those from the noncomplete chemotherapy patients; n=8). Taken together, the inventors of the instant application found that there was a distinguishable miRNA expression signature between the chemotherapy-responsive and chemotherapy-resistant EOC patients, and expression of the tumor suppressor miRNA Let-7i was significantly reduced in the chemotherapy-resistant EOC patients.

TABLE 1
Patient characteristics (N = 72)

| Characteristic | No. (%) | |
|---|---|---|
| Age | | |
| 20-29 | 1 | (0.01) |
| 30-39 | 3 | (0.04) |
| 40-49 | 10 | (0.14) |
| 50-59 | 23 | (0.32) |
| 60-69 | 20 | (0.28) |
| 70-79 | 14 | (0.19) |
| >80 | 1 | (0.01) |
| Stage | | |
| III | 61 | (0.85) |
| IV | 11 | (0.15) |
| Grade | | |
| 0 | 1 | (0.01) |
| 1 | 4 | (0.06) |
| 2 | 12 | (0.17) |
| 3 | 55 | (0.76) |
| Histologic subtypes | | |
| Serous | 41 | (0.57) |
| Endometrial | 6 | (0.08) |
| Mucinous | 7 | (0.10) |
| Clear cell | 4 | (0.06) |
| Others | 14 | (0.19) |
| Debulking status* | | |
| Optimal (V1 cm) | 23 | (0.32) |
| Suboptimal (>1 cm) | 48 | (0.67) |
| Chemotherapy response^{†} | | |
| Complete response | 42 | (0.58) |
| Noncomplete response | 27 | (0.38) |

*One patient not available.
^{†}Three patients not available.

### Example 2

### Decreased Let-7i Expression Increases the Chemotherapy Resistance of EOC Cells

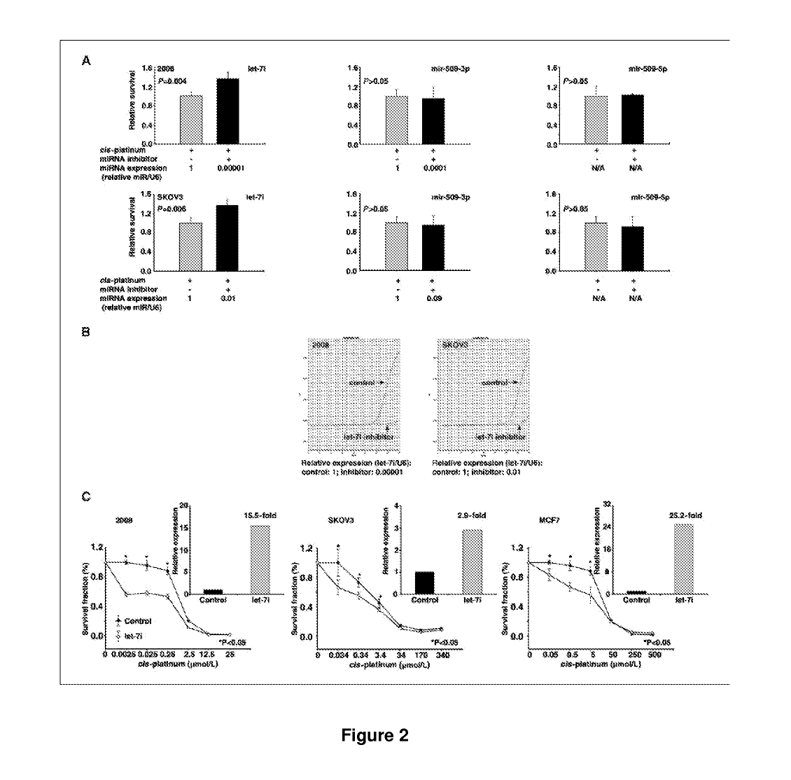

It was demonstrated that miRNAs are globally down-regulated in human cancers including EOC. Those down-regulated miRNAs, such as the Let-7 family, might serve as tumor suppressor genes and their suppression can have an important effect on tumor cells, e.g., by rendering them more resistant to cytotoxic anticancer therapy. Let-7i has been reported to be down-regulated in recurrent ovarian tumors compared with primary tumors. Therefore, to further investigate whether the above identified miRNAs are functionally involved in tumor resistance to chemotherapy, three miRNAs (Let-7i, mir-321, and mir-509; FIG. 1A) that were significantly repressed in the chemotherapy resistant tumors were focused on. mir-321, a fragment of Arg-tRNA, was excluded from the study, and both mature forms of mir-509 (mir-509-5p and mir-509-3p) were included. A total of three mature miRNAs, Let-7i, mir-509-5p, and mir-509-3p were examined in EOC cell lines (2008 and SKOV3) in vitro. Endogenous miRNA expression was blocked by specific antisense oligonucleotide inhibitors. The effect on miRNA expression by the inhibitor was confirmed by stem-loop real-time reverse transcription-PCR. More than 90% of the endogenous miRNA expression was blocked by the inhibitor 48 hours post transfection (FIG. 2). It was found that knockdown of the Let-7i expression, but not that of mir-509-3p or mir-509-5p, significantly increased cell resistance to cis-platinum treatment in various EOC cell lines (2008, P=0.004; SKOV3, P=0.006; FIG. 2A). A similar result was also found in short-term primary cultured ovarian tumor cells. To complement this loss-of-function study, Let-7i expression was stably enforced in EOC (2008 and SKOV3) and breast (MCF7) cell lines via retroviral transduction before exposing them to serial concentrations of cis-platinum. Overexpression of Let-7i in each of the above cell lines was confirmed by stem-loop real-time reverse transcription-PCR (FIG. 2C). Consistent with the loss-of-function study, overexpression of Let-7i significantly increased the chemotherapy response sensitivity in vitro (FIG. 2C). Taken together, down-regulated or intrinsically reduced Let-7i expression could render EOC cells more resistant to the cis-platinum treatment. Therefore, Let-7i serves as an important chemotherapy response modulator in cancer cells.

### Example 3

### Let-7i DNA Copy Number Does Not Exhibit Genomic Alteration in Human Cancer

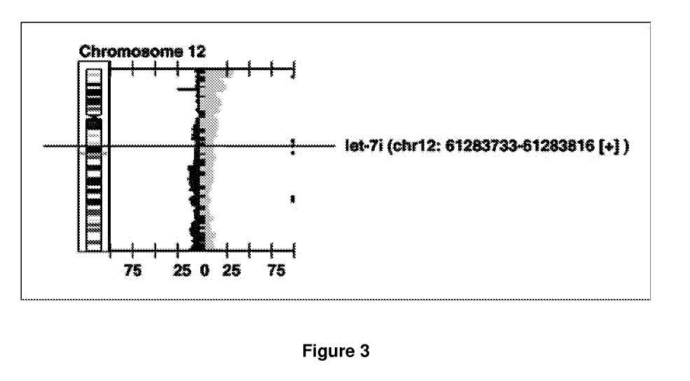

The molecular mechanism of Let-7i downregulation in patients with chemotherapy-resistant EOC is unclear. Previous studies indicated that DNA copy number of miRNAs is highly altered in human cancer including EOC, and DNA copy number alteration significantly contributes to miRNA expression in cancer. For example, Let-7a3 deletion was found in 31.2% of EOC specimens (n=106), which significantly reduced Let-7a3 expression in EOC. Therefore, the inventors of the instant application questioned whether DNA copy alteration of Let-7i contributes to the reduced expression of Let-7i in patients with chemotherapy-resistant EOC. In the 69 patients that were used for initial analysis of chemotherapy-associated miRNA markers, 30 were analyzed by aCGH (complete response patients, n=20; and noncomplete response patients, n=10). The inventors of the instant application first analyzed the genomic locus, Chr12_{—}61-62 Mb, which contains the primary Let-7i gene sequence, in these specimens. However, there was only one patient with a Let-7i DNA copy number alteration in the chemotherapy response group ( 1/30, 3.3%), no patients with either deletion or application were found in the chemotherapyresistant group. This shows that unlike other Let-7 family members, Let-7i does not significantly exhibit DNA copy alteration in EOC. Therefore, DNA copy number alteration might not affect Let-7i reduced expression in patients with chemotherapy-resistant EOC. For future confirmation of this conclusion, the inventors of the instant application expanded their aCGH study to a large collection of specimens with multiple cancer types including nine different types of human solid tumors (bladder breast, colon, lung, ovarian and pancreatic cancer, sarcoma, neuroblastoma, and melanoma; n=1,315; FIG. 3). Consistent with the first analysis, the DNA copy number of Let-7i was found in only extremely low frequency alterations (gained three to five copies in 5% and heterogeneously deleted in 6%, <10% alteration was usually considered as the background signal of aCGH), which was significantly lower than other members of the Let-7 family (e.g., Let-7a-3 and Let-7b deleted in 31.2% of EOC). These results show that other unknown mechanisms reduced Let-7i expression in the chemotherapy resistant patients, e.g., mutation, miRNA biogenesis pathway, epigenetic, or transcriptional regulation.

### Example 4

### Low let-7i Expression Is Significantly Associated with Shorter Survival of Patients with EOC

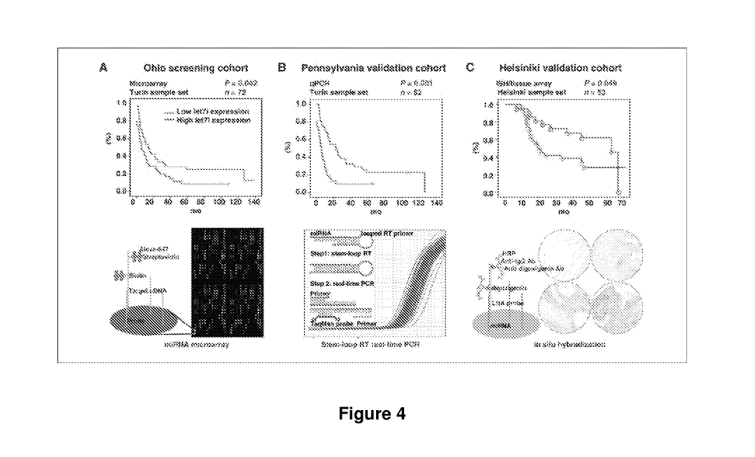

It has been reported that the expression of Let-7 family is a strong prognostic marker for human cancer patients. In this study, the inventors of the instant application identified Let-7i as an important predictor for chemotherapy resistance in patients with EOC. The inventors of the instant application further investigated whether Let-7i could also serve as a prognostic marker in patients with EOC. To examine the correlation between Let-7i expression and rapid recurrence of the disease, the Let-7i expression was studied in 72 late-stage EOC patient samples by miRNA microarray. Kaplan-Meier survival analysis indicated that low expression of Let-7i was significantly associated with shorter progression-free survival of the patients as compared with the high Let-7i expression group (P=0.042, n=72; FIG. 4A). This result was further validated by a more accurate mature miRNA quantitative method in the same sample set. Consistently, a similar result was also observed in the 62 randomly selected EOC patient samples analyzed by stem-loop real-time reverse transcription-PCR (n=62, P=0.001; FIG. 4B). Finally, Let-7i expression was analyzed in an independent sample set using a completely different methodology—in situ hybridization. Again, the inventors of the instant application found that lower Let-7i expression was significantly associated with shorter disease-free survival in 53 samples examined by in situ hybridization of tissue array (n=53, P=0.049; FIG. 4C). In conclusion, the above data clearly shows that the expression level of Let-7i could serve as a novel prognostic and prediction biomarker for the survival of patients with EOC.

### Example 5

### Cancer Treatment

Let-7 mimic and control oligos were purchased from Ambion/ABI. Cells were seeded in 24 or 96-well plates in antibiotic-free media to reach 40-50% confluence overnight. Twenty-four hours later, mimic delivery was performed using Lipofectamine RNAi Max Transfection Reagent (Invitrogen). Dose and time-dependent experiments was performed in vitro, exposing cells to 1 nM, 5 nM, 10 nM, 50 nM, 100 nM or 150 nM mimic for 24 hrs, 48 hrs, 72 hrs or 96 hrs. Total RNA are isolated from cells with TRIzol reagent (Invitrogen). The quality and quantity of the RNA are analyzed using a Bioanalyzer 2100 system. The effects of miRNA mimics on the expression of mature miRNA was examined by RT-PCR.

### Let-7 Mimic Treatment Inhibits Tumor Cell Growth in Vitro

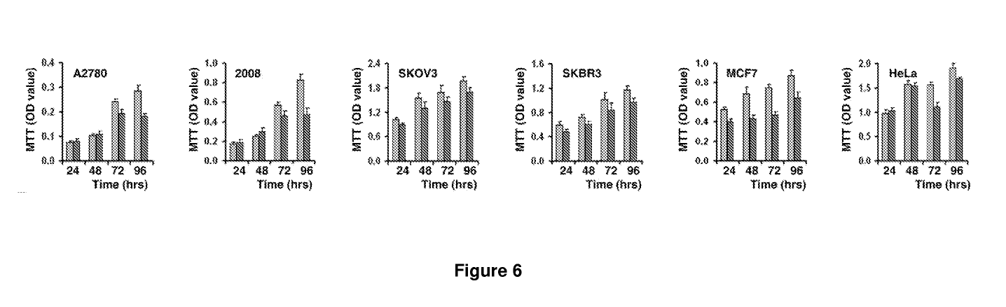

Tumor cell lines (A2780, 2008, SKOV3 (ovarian); SKBR3, MCF7 (breast); and HeLa (cervical).) were treated with let-7 mimic or control oligos in vitro. Cell growth was measured by MTT assay (Roche). As shown in FIG. 6, the proliferating rates of the let-7 mimic treated cells (red/black) were significantly lower than the control cells (green/grey) after 72 hrs.

This result clearly shows that let-7 replacement therapy along can block tumor cell growth.

### Let-7 Mimic Treatment Increases Chemotherapy Sensitivity in Vitro

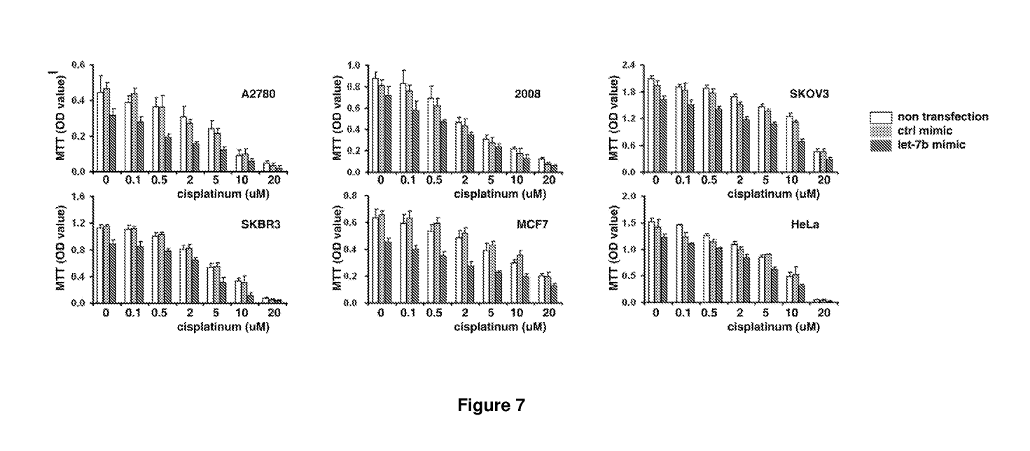

Tumor cell lines (A2780, 2008, SKOV3 (ovarian); SKBR3, MCF7 (breast); and HeLa (cervical).) were treated with chemotherapy drug cisplatinum and let-7 mimic or control oligos in vitro. Cell growth was measured by MTT assay (Roche). As shown in FIG. 7, the combination therapy (platinum+let-7 mimic) significantly reduced tumor cell growth.

This result clearly shows that let-7 replacement therapy can serve as modifier for chemotherapy.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A method for determining a chemotherapy response to treat a cancer, in a subject, comprising the steps of: obtaining a biological sample from said subject; and testing said biological sample to determine whether or not a microRNA (miRNA) is under-expressed in said biological sample, relative to the expression of said miRNA in a control sample, whereby the under-expression of said miRNA in said biological sample indicates a tumor response to said chemotherapy.

2. The method of claim 1, whereby said miRNA is a Let-7 miRNA.

3. The method of claim 2, whereby said Let-7 miRNA is Let-7i.

4. The method of claim 1, whereby the tumor response is a tumor resistance to said chemotherapy.

5. The method of claim 1, whereby said cancer is an ovarian cancer

6. The method of claim 1, whereby said cancer is an epithelial ovarian cancer.

7. The method of claim 1, whereby the step of testing said biological sample comprises analyzing a high-throughput expression of a plurality of miRNA.

8. The method of claim 7, whereby the high-throughput expression is detected from an array.

9. The method of claim 8, whereby said array is a micro-array of said plurality of miRNA.

10. The method of claim 1, whereby the step of testing said biological sample comprises analyzing an in situ hybridization of said miRNA in a cell of said biological sample.

11. The method of claim 1, whereby the step of testing said biological sample comprises analyzing a northern-blot expression of said miRNA.

12. The method of claim 1, whereby the step of testing said biological sample comprises analyzing a detectably labeled oligonucleotide complementary to said miRNA.

13. The method of claim 1, whereby said chemotherapy comprises treating with a cis-platinum.

14. The method of claim 1, whereby said biological sample comprises tumor cells.

15. The method of claim 1, whereby said biological sample is a tumor tissue.

16. A method for determining a chemotherapy response to treat an ovarian cancer, in a subject, comprising the steps of: obtaining a biological sample from said subject; and testing said biological sample to determine whether or not Let-7i miRNA is under-expressed in said biological sample, relative to the Let-7i miRNA expression in a control sample, whereby the under-expression of Let-7i miRNA in said biological sample indicates that an ovarian tumor in said subject is resistant to said chemotherapy.

17. A method for diagnosis of a cancer, in a subject, the method comprising the steps of:
obtaining a biological sample from said subject; and testing said biological sample to determine whether or not a microRNA (miRNA) is under-expressed in said sample, relative to the expression of said miRNA in a control sample, whereby the under-expression of said miRNA in said biological sample indicates that a tumor in said subject is resistant to a chemotherapy.

18. The method of claim 17, whereby said miRNA is a Let-7 miRNA.

19. The method of claim 18, whereby said Let-7 miRNA is Let-7i.

20. The method of claim 17, whereby said cancer is an ovarian cancer.

21. The method of claim 17, whereby said cancer is an epithelial ovarian cancer.

22. The method of claim 17, whereby the step of testing said biological sample comprises analyzing a high-throughput expression of a plurality of miRNA.

23. The method of claim 22, whereby the high-throughput expression is detected from an array.

24. The method of claim 23, whereby said array is a micro-array of said plurality of miRNA.

25. The method of claim 17, whereby the step of testing said biological sample comprises analyzing an in situ hybridization of said miRNA in a cell of said biological sample.

26. The method of claim 17, whereby the step of testing said biological sample comprises analyzing a northern-blot expression of said miRNA.

27. The method of claim 17, whereby the step of testing said biological sample comprises
analyzing a detectably labeled oligonucleotide complementary to said miRNA.

28. The method of claim 17, whereby said chemotherapy comprises treating with a cis-platinum.

29. The method of claim 17, whereby said biological sample comprises tumor cells.

30. The method of claim 17, whereby said biological sample is a tumor tissue.

31. A method of providing a prognosis for a cancer, in a subject, the method comprising the steps of: obtaining a biological sample from said subject; and testing said biological sample to determine whether or not a microRNA (miRNA) is under-expressed in said sample, relative to the expression of said miRNA in a control sample, whereby the under-expression of said miRNA in said biological sample indicates that a tumor in said subject is resistant to a chemotherapy.

32. A method of improving a chemotherapy response to a cancer treatment, in a subject, the method comprising administering an effective amount of an agent that enhances the expression of a microRNA (miRNA).

33. The method of claim 32, whereby said miRNA is a Let-7 miRNA.

34. The method of claim 33, whereby said Let-7 miRNA is Let-7i.

35. The method of claim 32, whereby said agent is a shRNA from a polymerase II or III promoter.

36. The method of claim 32, whereby said agent is a double-stranded miRNA mimic.

37. The method of claim 32, whereby said agent is an oligonucleotide based pre-mir-Let-7 drug.

38. The method of claim 32, whereby said cancer is an ovarian cancer

39. The method of claim 32, whereby said cancer is an epithelial ovarian cancer.

40. The method of claim 32, whereby said chemotherapy comprises treating with a cis-platinum.

41. A method of treating a cancer, in a subject, the method comprising: administering an effective amount of a chemotherapy agent and an effective amount of an agent that enhances the expression of a microRNA (miRNA).

42. A kit for determining a chemotherapy response in a patient with a cancer, said kit comprising: a) a oligonucleotide complementary to an miRNA; and b) optionally, reagents for the formation of the hybridization between said oligonucleotide and said miRNA.

43. The kit according to claim 42, wherein said miRNA is a Let-7 miRNA.

44. The kit according to claim 43, wherein said Let-7 miRNA is Let-7i.

45. The kit according to claim 42, wherein said miRNA is detectably labeled.

46. The kit according to claim 42, wherein said miRNA is attached to a solid surface.

47. The kit according to claim 42, wherein said miRNA is a member of a nucleic acid array.

48. The kit according to claim 47, wherein said nucleic acid array is a micro-array.

49. An apparatus for determining a chemotherapy response in a patient with a cancer, said apparatus comprising a solid support, wherein a surface of said solid support is linked to an oligonucleotide complementary to an miRNA.

50. A pharmaceutical composition for improving a tumor response to chemotherapy, said composition comprising an effective amount of an agent that enhances the expression of an miRNA in said tumor.
